(19) United States
(12) Patent Application Publication
(10) Pub. No.: US 2023/0013852 A1
(43) Pub. Date: Jan. 19, 2023

(54) METHOD AND APPARATUS FOR STERILIZING MEDICAL INSTRUMENTS

(71) Applicant: Log10 B.V., Eindhoven (NL)

(72) Inventors: Franciscus Maria VERHOEVEN, Amsterdam (NL); Theo Alex Eduard VAN DER LEIJ, Moergestel (NL); Mirte PEETERS, Dongen (NL); Thijs DE JONG, de Bilt (NL)

(21) Appl. No.: 17/783,057

(22) PCT Filed: Dec. 8, 2020
(86) PCT No.: PCT/NL2020/050766

§ 371 Date: Jun. 7, 2022

(30) Foreign Application Priority Data
Dec. 9, 2019 (NL) 2024409

(51) Int. Cl. A61L 2/14; A61L 2/24

(52) U.S. Cl. CPC A61L 2/14; A61L 2/24; A61L 2202/24; A61L 2202/122; A61L 2202/17; A61L 2202/14

(57) ABSTRACT

A method and apparatus for sterilizing a medical instrument, such as a dental instrument. The method includes placing the medical instrument in a chamber, providing a sterilizing agent including recombined ionized humidified air, controlling the temperature of the medical instrument and/or the chamber such that the temperature of the medical instrument is below the temperature of chamber, and at least partially condensing the sterilizing agent onto the instruments.

### FIELD OF THE INVENTION

The present invention relates to sterilizing medical instruments, such as dental instruments.

### BACKGROUND TO THE INVENTION

Reusable medical instruments are instruments that health care providers can reuse to diagnose and/or treat multiple patients. Examples of reusable medical instruments include medical instruments used in dental care, such as scalpels, syringes, scopes, mirrors, drills, burs, discs, handpieces, excavators, turbines, files, reamers, etc.

When used on patients, reusable instruments become soiled and contaminated with blood, tissue and other biological debris such as microorganisms. To avoid any risk of infection by a contaminated instrument, the reusable instruments can be sterilized. Sterilizing results in a medical instrument that can be safely used more than once in the same patient, or in more than one patient. Adequate sterilizing of reusable medical instruments is vital to protecting patient safety.

Various sterilizing agents can be used for sterilizing medical instruments. Historically, steam and/or hydrogen peroxide is often used. More recently, plasma devices are being used for ionizing gases or gas mixtures, the ionized gas being used as sterilizing agent. Electrons in the plasma impact on gas molecules causing dissociation and ionization of these molecules, which creates a mix of reactive species. It is known to directly expose the medical instruments to the plasma, or to expose the medical instruments to the (partially) recombined plasma, sometimes referred to as afterglow, see e.g. S. Moreau et al., “Using the flowing afterglow of a plasma to inactivate Bacillus subtilis spores: Influence of the operating conditions”, J. Appl. Phys. Vol. 88, No. 2, 15 Jul. 2000.

Several attempts have been made to improve upon plasma sterilizing. US2011/0027125A1 discloses a system comprising a chamber and a plasma generator for generating free radicals combined with use of a hydrogen peroxide solution.

There exists a need for more efficient and effective plasma sterilizing.

### SUMMARY OF THE INVENTION

It is an object to provide a method and system for sterilizing medical instruments, such as dental instruments. It is an object to provide a more efficient and/or effective method and system for sterilizing medical instruments, such as dental instruments.

Thereto, according to an aspect is provided a method for sterilizing a medical instrument, such as a dental instrument. The method includes placing the medical instrument, or a plurality of medical instruments, in a chamber. The medical instrument is preferably dry, or dried e.g. after preceding rinsing and/or washing steps, before sterilizing. The method includes providing a sterilizing agent including recombined ionized humidified air, i.e. humidified air that has been ionized and allowed to at least partially recombine. The recombined ionized humidified air can be obtained by treating air with a plasma device, e.g. feeding an air stream through a plasma source, and allowing the ionized air to at least partially recombine. The air can be humidified prior to feeding the air to the plasma source, e.g. for obtaining a well-defined humidity of the air. Herein “air” refers to ambient air or air-like gas mixtures, such as a mixture of nitrogen and oxygen gas, possibly with further additions such as carbon dioxide gas. The method includes controlling the temperature of the medical instrument and/or the chamber such that the temperature of the medical instrument is below the temperature of chamber. Ensuring that the temperature of the instrument is lower than the temperature of the chamber allows condensing the sterilizing agent onto the instrument, while condensing of sterilizing agent onto the, e.g. walls of, the chamber can be prevented. Preferably the temperature of the medical instrument and the temperature of the chamber walls are chosen such that the sterilizing agent condenses onto the instrument, while condensing of sterilizing agent onto the walls of the chamber is be prevented. Thus, efficient use is made of the sterilizing agent for sterilizing the instrument, instead of for sterilizing the chamber. Thereto, the temperature of the medical instrument, the temperature of the chamber and the humidity of the sterilizing agent can be chosen appropriately. The condensing of the sterilizing agent onto the instrument has a beneficial effect on the effectivity of the sterilizing. Without wishing to be bound to any theory, it is believed that the condensate allows for proper covering of the entire surface of the instrument with sterilizing agent, as well as providing a synergistic effect between the active ingredients of the sterilizing agent with water in the condensate.

Optionally the medical instrument is cooled. Cooling the medical instrument to a temperature below the temperature of the chamber can be achieved by simple means. The medical instrument can also be maintained at a temperature below the temperature of the chamber, e.g. by cooling the medical instrument to prevent the medical instrument from being heated by the chamber.

Optionally, the medical instrument is cooled prior to placing the medical instrument in the chamber. This way the cooling can be simplified as cooling is not confined to the limited space of the chamber. The medical instrument can e.g. be cooled in a cooling chamber prior to being transferred to the chamber for sterilization.

Optionally, the medical instrument is cooled inside the chamber. Hence, a simple method can be provided wherein the medical instrument does not require a thermal pre-treatment, but can simply be inserted into the chamber. Also undesired condensation onto the medical instrument prior to exposing the medical instrument to the sterilizing agent can be avoided.

Optionally, the walls of the chamber are heated to above the temperature of the medical instrument. By actively heating the walls of the chamber, the temperature of the chamber becomes independent of ambient temperature. Also, by controlling the temperature of the chamber condensation of the sterilizing agent onto the walls of the chamber can be prevented more effectively and/or efficiently. The chamber can also be maintained at a temperature above the temperature of the medical instrument, e.g. by heating the chamber to prevent the chamber from being cooled by the medical instrument.

The temperature of the instrument is lower than the temperature of the chamber, at least when starting to supply the sterilizing agent to the chamber, preferably during the entire sterilizing operation. Optionally, the medical instrument is cooled or maintained below ambient temperature. Optionally, the walls of the chamber are heated or maintained above ambient temperature. The medical instrument can e.g. be cooled or maintained below. The walls of the chamber can e.g. be heated or maintained above 25° C. Optionally, the medical instrument is cooled to below the dew point of at least one of the components of the sterilizing agent. This can further enhance condensing of the sterilizing agent onto the medical instrument. Optionally, the walls of the chamber are heated or maintained above the dew point of at least one of the components of the sterilizing mixture. The at least partly recombined ionized humidified air can include reactive oxygen and/or nitrogen species, e.g. produced in the plasma source. Hence, the sterilizing mixture can include one or more of the following components: O₂, O₂⁻, O., O₃, O, OH, H, H₂, HO₂, NOx, H₂O₂, and OH⁻.

Optionally, a temperature difference between the medical instrument and the walls of the chamber, at least at the onset of the sterilizing process, is more than about 5° C., such as more than about 10° C., e.g. more than about 15° C. Without wishing to be bound to any theory, it has been found that such temperature differences can have a beneficial effect on sterilizing efficiency and/or effectivity.

Optionally, the medical instrument, or a plurality of medical instruments, is placed in a container, and the container is placed in the chamber. The container can be arranged to be hermetically closed or at least provide a microbial barrier preventing microbes from leaving and/or entering the inner space of the container. Thus one or more contaminated medical instruments can easily be packaged, e.g. safe to personnel, and inserted into the chamber while in the container. The medical instruments can be sterilized inside the chamber while in the container. The container can be cooled to below the temperature of the chamber. Hence, the medical instruments in the container can easily be cooled together with the container. Also, hence the container, which can be contaminated as well, can easily be sterilized. Once sterilized, the container can be hermetically closed or at least closed to provide a microbial barrier preventing microbes from entering the inner space of the container, preferably while still inside the chamber. Thus, the sterilized medical instruments are packaged in the container for future use while remaining sterilized.

Optionally, the pressure inside the chamber is reduced prior to providing the sterilizing agent to the chamber. Thus, the sterilizing agent need not displace, or at least displace less, gas already present in the chamber. Hence, the sterilizing agent can efficiently reach the medical instrument to de sterilized.

Optionally, the medical instrument is cooled using a gas or gas mixture, such as air. The gas can be a cooled gas. The cooling can include subjecting the medical instrument to a stream of the gas. The cooling can include atomizing water into a cooling gas stream and impacting the atomized water and cooling gas stream onto the medical instruments. Thus, efficient cooling of the medical instrument can be achieved.

Optionally, the step of providing the sterilizing agent includes providing a plasma source, feeding a humidified air stream through the plasma source, at least partially ionizing the air stream, and allowing the air stream to at least partially recombine.

Optionally, the sterilizing is performed at or below ambient pressure. The sterilizing can e.g. be performed at about 800-1050 mbar. The plasma source can be an ambient pressure plasma source. The ambient pressure plasma source can e.g. be operated at a pressure of 800-1200 mbar.

Optionally, the method includes washing the medical instruments prior to sterilization. The cooling gas stream, e.g. including the atomized water, can be supplied to the washed medical instruments for drying and cooling the medical instruments. Thus dried and cooled medical instruments can be subjected to the sterilizing agent. It will be appreciated that simultaneously drying and cooling the medical instrument with the air stream including atomized water can also separately be used in other types of sterilizing or disinfecting medical instruments, such as dental instruments.

According to an aspect is provided an apparatus for sterilizing a medical instrument. The apparatus includes a chamber arranged for placing the medical instrument, or a plurality of medical instruments, therein. The apparatus includes a sterilizing agent source arranged for providing a sterilizing agent including recombined ionized humidified air. The apparatus includes a temperature control unit arranged for controlling the temperature of the medical instrument and/or the chamber such that the temperature of the medical instrument is below the temperature of chamber. As explained above, by having the instruments at a temperature below the temperature of the chamber, e.g. below the temperature of walls of the chamber, condensing of sterilizing agent onto the, e.g. walls of, the chamber can be prevented while condensing of at leas a component of the sterilizing agent onto the medical instrument can be promoted. Thus, efficient use is made of the sterilizing agent for sterilizing the instrument, instead of for sterilizing the chamber.

Optionally, the temperature control unit includes a cooling unit arranged for cooling the medical instrument to below temperature of chamber for allowing the sterilizing agent to condense onto the instrument. The cooling unit can also be arranged for maintaining the temperature of the medical instrument below the temperature of the chamber.

Optionally, the cooling unit is arranged for cooling the medical instrument prior to placing the medical instrument in the chamber. Optionally, the cooling unit is arranged for cooling the medical instrument inside the chamber.

Optionally, the cooling unit is arranged for controlling the temperature of the medical instrument, e.g. to a predetermined temperature. Thereto, the cooling unit can include a temperature sensor and a controller.

Optionally, the temperature control unit includes a heating unit arranged for heating the walls of the chamber above the temperature of the medical instrument. The heating unit can also be arranged for maintaining the temperature of the chamber above the temperature of the medical instrument.

Optionally, the apparatus further includes a container arranged for receiving the medical instrument, and arranged for being placed in the chamber. The cooling unit can be arranged for cooling the container to below the temperature of the chamber.

Optionally, the apparatus includes a pump arranged for reducing the pressure inside the chamber prior to providing the sterilizing agent to the chamber.

Optionally, the cooling unit includes a gas conduit for cooling the medical instrument using a gas, such as air. The gas conduit can include a mouth, such as one or more nozzles, pointing a stream of the gas onto the medical instrument an/or container. The cooling unit can include an atomizer for atomizing water into a cooling gas stream and impacting the atomized water and cooling gas stream onto the medical instruments.

The apparatus can include a plasma source, having an input port for feeding a humidified air stream into the plasma source, and an output port for feeding the air stream to the chamber while allowing the air stream to at least partially recombine.

Optionally, the apparatus further includes a washing unit arranged for washing and/or rinsing the medical instruments prior to sterilization. The cooling gas stream including the atomized water is can be supplied to the washed medical instruments for drying and cooling the medical instruments.

It will be appreciated that any of the aspects, features and options described in view of the method apply equally to the apparatus. It will also be clear that any one or more of the above aspects, features and options can be combined.

### BRIEF DESCRIPTION OF THE DRAWINGS

Embodiments of the present invention will now be described in detail with reference to the accompanying drawings in which:

FIG. 1 shows a schematic representation of an example of an apparatus;

FIG. 2 shows a schematic representation of a flow chart;

FIG. 3 shows a schematic representation of an example of an apparatus; and

FIG. 4 shows a schematic representation of an example of an apparatus.

### DETAILED DESCRIPTION

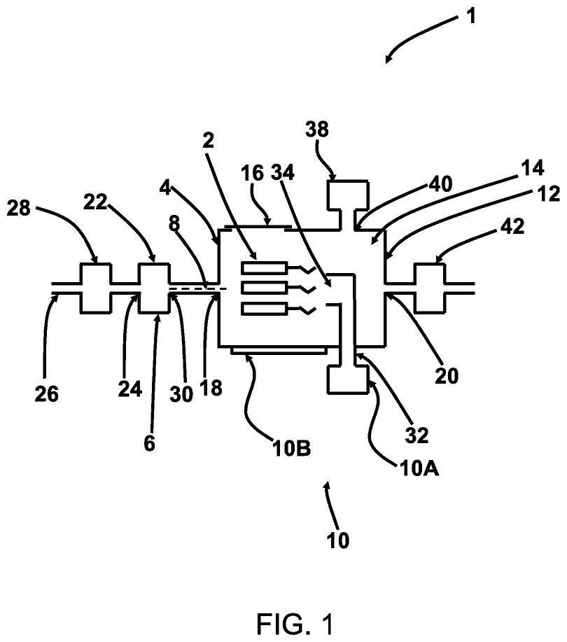

FIG. 1 shows a schematic representation of an example of an apparatus 1 for sterilizing a medical instrument 2. The apparatus 1 includes a chamber 4. The chamber 4 is arranged for placing the medical instrument 2 to be sterilized therein. In this example, the chamber 4 is arranged for placing a plurality of medical instruments 2 to be sterilized therein. The apparatus 1 includes a sterilizing agent source 6. The sterilizing agent source 6 is arranged for providing a sterilizing agent 8. The sterilizing agent 8 includes recombined ionized humidified air. The apparatus 1 includes a temperature control unit 10. In this example, the temperature control unit 10 includes a cooling unit 10A. The cooling unit 10A is arranged for cooling the medical instruments 2.

The chamber 4 includes walls 12 forming an internal space 14 for receiving the medical instruments 2. In this example, the chamber 4 has a door 16 for allowing the medical instruments 2 to be inserted into and extracted from the internal space 14 of the chamber 4. The chamber 4 includes a sterilizing agent supply port 18. The chamber 4 includes an exhaust port 20.

The sterilizing agent source 6 here includes a plasma source 22. The plasma source 6 includes an input port 24 for feeding a humidified air stream into the plasma source 6. In FIG. 1 the input port 24 is connected to a an air stream supply 26 via a humidifier 28. The plasma source 6 includes an output port 30 in communication with the sterilizing agent supply port 18 of the chamber 4.

The cooling unit 10A in this example includes a gas conduit 32 for cooling the medical instrument using a gas, here air. The gas conduit 32 includes a mouth 34, here nozzles, pointing a stream of the gas onto the medical instrument 2.

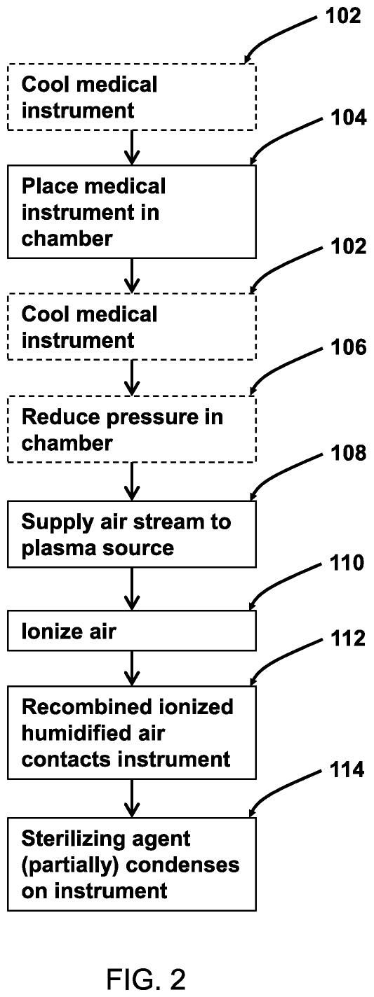

The apparatus 2 can be used as follows. FIG. 2 shows a schematic representation of a flow chart of a method for sterilizing a medical instrument. In a first step 102 medical instruments 2 are cooled by activating the cooling unit 10 and having the stream of cooling gas impact and cool the instruments 2. The instruments 2 to be sterilized are placed in the internal space 14 of the chamber 4 in step 104. The step 104 can be performed before step 102, after step 102, or both before and after step 102. The medical instruments 2 are cooled to below the temperature of the chamber 4, here to below the temperature of the walls 12 of the chamber 4. The temperature control unit 10 can include a heating unit 10B arranged for heating or maintaining the walls 12 of the chamber 4 above the temperature of the medical instrument 2, or above the ambient temperature. In this example, the pressure in the chamber 4 is reduced 106, e.g. to about 100 mbar. Thereto a pump 38 connected to a pumping port 40 of the chamber 4 can be activated.

An air stream is supplied 108 to the input port 24 of the plasma source 6 via the humidifier 28. Depending on the humidity of the air supplied to the humidifier, the humidifier can add or remove water from the air such that at the exit of the humidifier an air stream with a predetermined humidity is obtained. In this example, the air stream entering the plasma source has a predetermined specific humidity, SH. The specific humidity of the air entering the plasma source 6 can e.g. be 10±1 g/kg (grams of water per kg of air). In the plasma source 6 the air is ionized 110. The ionized air is fed from the plasma source 6 into the sterilizing agent supply port 18 of the chamber 4. During transport the ionized humidified air, at least partly, recombines. The sterilizing agent formed by the at least partly recombined ionized humidified air then contacts 112 the medical instrument to be sterilized. As the medical instrument had been cooled, the sterilizing agent, at least partially, condenses 114 onto the medical instrument and sterilizes the medical instrument. As the walls 12 of the chamber 4 are not cooled, less cooled than the medical instruments, or even heated, condensation of the sterilizing agent onto the walls 12 can be prevented. After sterilization the sterilizing agent can be removed from the chamber 4 via the exhaust port 20. A destructor 42 may be placed in communication with the exhaust port 20 for destructing any contaminants carried by the exhausted sterilizing agent.

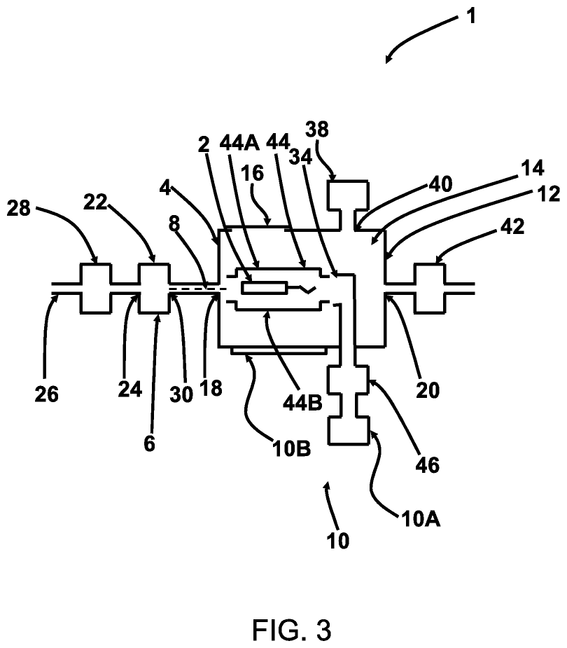

FIG. 3 shows a schematic representation of an example of an apparatus 1 for sterilizing a medical instrument 2. The example of FIG. 3 is similar to the example of FIG. 1. A main difference is that the apparatus 1 of FIG. 3 further includes a container 44. The container 44 is arranged for holding the medical instrument 2, here for holding a plurality of medical instruments 2. The container in this example includes a tray 44A and a lid 44B. The container 44 can be opened by removing the lid 44B from the tray 44A for placing one or more medical instruments 2 inside the container 44. The container 44 is arranged for being placed in the chamber 4. The chamber 4 can include guides for holding the container 44. The apparatus 1 in this example is arranged for opening the container inside the chamber 4. In the example of FIG. 3 the cooling unit 10 is arranged for cooling the container 44 to below the temperature of the chamber 4. Hence, the medical instruments 2 in the container 44 can easily be cooled together with the container 44. Also, hence the container 44, which can be contaminated as well, can easily be sterilized. After sterilization, the container 44 is closed, preferably while still inside the chamber 4. The container 44 can be arranged to be closed hermetically, or at least provide a microbial barrier preventing microbes from entering the inner space of the container.

In the example of FIG. 3 the cooling unit 10 includes an atomizer 46 for atomizing water into a cooling gas stream and impacting the atomized water and cooling gas stream onto the medical instruments 2 and/or onto the container 44.

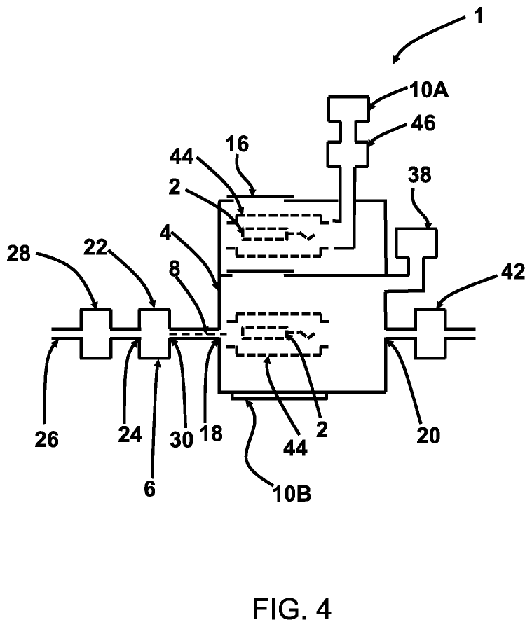

FIG. 4 shows a schematic representation of an example of an apparatus 1 for sterilizing a medical instrument 2. The example of FIG. 4 is similar to the example of FIG. 3. A main difference is that the apparatus 1 of FIG. 4 further includes a cooling chamber 46. The cooling chamber 46 is arranged for holding the medical instrument 2, here for holding the container 44 holding medical instruments 2 while cooling the medical instrument(s) 2 and optionally the container 44. In this example the medical instruments 2, here in the container 44, are cooled in the cooling chamber 44 and then transferred to the chamber 4 for sterilization. The apparatus 1 can include a handler unit for transferring the medical instruments 2 and/or the container 44 from the cooling chamber 46 to the sterilization chamber 4 after cooling.

Herein, the invention is described with reference to specific examples of embodiments of the invention. It will, however, be evident that various modifications and changes may be made therein, without departing from the essence of the invention. For the purpose of clarity and a concise description features are described herein as part of the same or separate embodiments, however, alternative embodiments having combinations of all or some of the features described in these separate embodiments are also envisaged.

In the example of FIG. 1 the cooling unit is arranged for cooling the medical instrument inside the chamber. It is also possible that alternatively, or additionally, the cooling unit is arranged for cooling the medical instrument prior to placing the medical instrument in the chamber.

In the example of FIG. 3 the cooling unit includes an atomizer. It will be appreciated that the cooling unit of FIG. 1 can also include an atomizer. Also, the atomizer can be omitted from the cooling unit of FIG. 3.

In the example of FIG. 4 the apparatus includes a cooling chamber. It will be appreciated that the cooling chamber can also be used in the apparatus of FIG. 1.

The walls of the chamber are not cooled, less cooled than the medical instruments, or even heated. Thereto a heating unit is described in view of the examples. It will be appreciated that the apparatus can also include a chamber cooling unit arranged for cooling the chamber to a temperature above the temperature of the medical instrument. It will be clear that it is also possible that the apparatus includes an instrument heating unit arranged for heating the medical instrument to a temperature below the temperature of the chamber.

It is possible that the apparatus further includes a washing unit arranged for washing and/or rinsing the medical instruments prior to sterilization. Preferably, the medical instruments are dried prior to sterilization. The cooling gas stream, optionally including the atomized water, can be supplied to the washed medical instruments for drying and cooling the medical instruments.

However, other modifications, variations, and alternatives are also possible. The specifications, drawings and examples are, accordingly, to be regarded in an illustrative sense rather than in a restrictive sense.

For the purpose of clarity and a concise description features are described herein as part of the same or separate embodiments, however, it will be appreciated that the scope of the invention may include embodiments having combinations of all or some of the features described.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. A method for sterilizing a medical instrument, including:
placing the medical instrument in a chamber;
providing a sterilizing agent including recombined ionized humidified air;
controlling the temperature of the medical instrument and/or the chamber such that the temperature of the medical instrument is below the temperature of chamber; and
at least partially condensing the sterilizing agent onto the instruments.

2. The method according to claim 1, including cooling or maintaining the medical instrument below the temperature of the chamber.

3. The method according to claim 2, including cooling the medical instrument prior to placing the medical instrument in the chamber.

4. The method according to claim 2, including cooling the medical instrument inside the chamber.

5. The method according to claim 1, including heating or maintaining the walls of the chamber above the temperature of the medical instrument.

6. The method according to claim 1, including cooling or maintaining the medical instrument to below the dew point of at least one of the components of the sterilizing agent.

7. The method according to claim 1, including heating or maintaining the walls of the chamber above the dew point of at least one of the components of the sterilizing mixture.

8. The method according to claim 1, wherein the medical instrument is placed in a container, and the container is placed in the chamber.

9. The method according to claim 8, including cooling the container to below the temperature of the chamber.

10. The method according to claim 1, including reducing the pressure inside the chamber prior to providing the sterilizing agent to the chamber.

11. The method according to claim 1, including cooling the medical instrument using a gas or gas mixture, such as air.

12. (canceled)

13. The method according to claim 1, wherein the step of providing the sterilizing agent includes:
providing a plasma source;
feeding a humidified air stream through the plasma source,
at least partially ionizing the air stream; and
allowing the air stream to at least partially recombine.

14. The method according to claim 1, wherein the sterilizing is performed at or below ambient pressure.

15. The method according to claim 1, including washing the medical instruments prior to sterilization.

16. The method according to claim 11, including washing the medical instruments prior to sterilization, wherein the cooling gas stream is supplied to the washed medical instruments for drying and cooling the medical instruments.

17. An apparatus for sterilizing a medical instrument, including:
a chamber arranged for placing the medical instrument therein;
a sterilizing agent source arranged for providing a sterilizing agent including recombined ionized humidified air; and
a temperature control unit arranged for controlling the temperature of the medical instrument and/or the chamber such that the temperature of the medical instrument is below the temperature of chamber for allowing the sterilizing agent to at least partially condense onto the instrument.

18. The apparatus of claim 17, wherein the temperature control unit includes a cooling unit arranged for cooling or maintaining the medical instrument below the temperature of the chamber.

19. (canceled)

20. (canceled)

21. The apparatus according to claim 17, wherein the temperature control unit includes a heating unit arranged for heating or maintaining the walls of the chamber above the temperature of the medical instrument.

22. The apparatus according to claim 17, further including a container arranged for receiving the medical instrument, and arranged for being placed in the chamber, wherein the cooling unit is arranged for cooling the container to below the temperature of the chamber.

23. (canceled)

24. (canceled)

25. The apparatus according to claim 17, wherein the cooling unit includes a gas conduit for cooling the medical instrument using a gas, such as air.

26. (canceled)

27. The apparatus according to claim 17, including a plasma source, having an input port for feeding a humidified air stream into the plasma source, and an output port for feeding the air stream to the chamber while allowing the air stream to at least partially recombine.

28. (canceled)

29. (canceled)
